(19) United States
(12) Patent Application Publication
(10) Pub. No.: US 2004/0072203 A1
(43) Pub. Date: Apr. 15, 2004

(54) Purification of primer extension products

(75) Inventors: Connie Kim Dix, Bothell, WA (US); Karin A. Hughes, Bothell, WA (US); Robert J. Kaiser, Bothell, WA (US); Mark L. Stolowitz, Woodinville, WA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Prolinx, Inc., Bothell, WA

(21) Appl. No.: 10/418,749

(22) Filed: Apr. 16, 2003

Related U.S. Application Data
(63) Continuation of application No. 09/350,053, filed on Jul. 8, 1999, now abandoned.
(60) Provisional application No. 60/125,611, filed on Mar. 19, 1999.

(51) Int. Cl. C12Q 1/68; C12M 1/34; C08G 63/91

(52) U.S. Cl. 435/6; 435/287.2; 525/54.2

(57) ABSTRACT

This invention provides methods for purifying nucleic acids, in particular primer extension products such as those obtained in nucleic acid sequencing reactions. The methods involve the use of a primer to which is attached a string of arylboronic acid moieties. After extension of the primer using a polymerase, the primer extension-products are complexed with a solid support to which is attached an arylboronic acid complexing moiety. The resulting complex is separated from the reaction mixture, washed, and the primer extension products are dissociated from the solid support. The primer extension products are obtained in a form particularly suitable for loading directly on a capillary electrophoresis apparatus.

### CROSS-REFERENCE TO RELATED APPLICATION

This application claims benefit of U.S. Provisional Application No. 60/125,611, filed Mar. 19, 1999, which application is incorporated herein by reference for all purposes.

### BACKGROUND OF THE INVENTION

The demands of the Human Genome Project and the commercial implications of polymorphism and gene discovery have driven the development of significant improvements in DNA sequencing technology. Contemporary approaches to DNA sequencing have imposed stringent demands on reliability and throughput for DNA sequencers. Recent reports have demonstrated the extraordinary potential of capillary electrophoresis (CE) for DNA sequencing given the inherent speed, resolving power and ease of automation associated with this method as compared to slab gel electrophoretic methods (Carrilho et al., Anal. Chem. 1996, 68, 3305-3313; Tan and Yeung, Anal. Chem. 1997, 69, 664-674; Swerdlow et al., Anal. Chem. 1997, 69, 848-855).

Relative to cross-linked gel capillary electrophoretic columns, the recent development of replaceable polymer solutions to achieve size separation of single-stranded DNA fragments has increased the lifetime of the columns and eliminated the requirements of gel pouring and casting (Ruiz-Martinez et al., Anal. Chem. 1993, 65, 2851-2858).

Additionally, improvements in the composition of the separation matrix have led to sequencing over 1000 bases per run (Carrilho et al., Anal. Chem. 1996, 68, 3305-3313).

Automated capillary electrophoresis systems for DNA sequencing have been introduced commercially by three major scientific instrument manufacturers (Beckman Coulter CEQ™ 2000 DNA Analysis System; Amersham Pharmacia MegaBACE 1000 DNA Sequencing System; and PE Biosystems ABI Prism 3700 DNA Analyzer).

Realizing the potential of this new generation of automated DNA sequencers is proving difficult, however, as problems in read length and accuracy remain, primarily due to the limitations associated with the methods currently available for purifying the products of sequencing reactions. Indeed, the critical importance of sample preparation for the successful implementation of capillary electrophoresis has not been sufficiently emphasized.

In contrast to slab gel electrophoresis, primer extension products are introduced into the capillary column using electrokinetic injection, which provides focusing of the single-stranded DNA fragments at the head of the column (Swerdlow et al., Proc. Natl. Acad. Sci. U.S.A. 1988, 85, 9660-966). However, electrokinetic injection is biased toward high electrophoretic mobility ions, such as chloride and dideoxynucleotides, which, if present in the sequencing reaction solution, negatively affect the focusing of single-stranded DNA fragments. Consequently, to increase the amount of DNA injected into the capillary column, and to improve the focusing of the injected DNA, an effective removal of these small ionic species is required.

The sample preparation scheme now routinely employed for both slab gel electrophoresis and CE consists of desalting DNA sequencing samples by ethanol precipitation, followed by reconstitution of the DNA fragments and template in a mixture of formamide-0.5 M EDTA (49:1) prior to loading or injection (Figeys et al., 1996, 744, 325-331; Sambrook, J.; Fritsch, E. F.; Maniatis, T. Molecular Cloning: A Laboratory Manual; Cold Spring Harbor Laboratory Press: Cold Spring Harbor, N.Y., 1989; section 9.49). Although widely utilized, this method has been found to exhibit variable reproducibility in terms of DNA recovery, and is not easily automated (Tan, H.; Yeung, E. S. Anal. Chem. 1997, 69, 664-674, and Hilderman, D.; Muller, D. Biotechniques 1997, 22, 878-879).

High electrophoretic mobility ionic species DNA sequencing samples are not the only contaminants that cause a degradation in sequencing read length. Template DNA also has been shown to interfere with the analysis of primer extension products in both thin slab gels (Tong et al., Biotechniques 1994, 16, 684-693), and capillary columns (Swerdlow et al., Electrophoresis 1996, 17, 475-483). Upon injection of the sequencing reaction solution, a current drop and significant deterioration in the resolving power of the capillary column is observed when template DNA is present in the sample (Salas-Solano et al., Anal. Chem. 1998, 70, 1528-1535). However, at present, template DNA removal is seldom considered an essential aspect of sample preparation for DNA sequencing by capillary electrophoresis.

Only two approaches to sample preparation that address the need for template removal have been proposed thus far. In the first approach, which is described in U.S. Pat. No. 5,484,701, a biotinylated primer enables the capture and purification of primer extension products on streptavidin magnetic particles. After extensive washing of the primer extension products immobilized on the streptavidin magnetic particles to remove the sequencing reaction constituents including template DNA, release of the primer extension products is effected by heating the streptavidin magnetic particles to from about 90° C. to 100° C. in a formamide solution.

Although this approach has considerable utility in conjunction with slab gel electrophoresis (in which formamide is often added to sequencing samples to facilitate denaturation of duplex DNA and to increase the viscosity of the sample to facilitate slab gel loading), it has recently been shown to be problematic when utilized in conjunction with capillary electrophoresis. At least three distinct problems (exclusive of cost) have been identified as being associated with this approach. First, the formamide solution utilized to effect release of immobilized primer extension products is incompatible with electrokinetic injection, owing to the high ionic strength of the solution due to the presence of high electrophoretic mobility ions (most notably 10 mM EDTA or 30-140 mM sodium acetate in 95% formamide). In the absence of salt in the formamide solution, the efficiency of release of biotinylated primer extension products has been shown to be significantly reduced from >95% to <40% (Tong and Smith, Anal. Chem. 1992, 64, 2672-2677). The effective ionic strength of the release solution has been shown to be still further increased by decomposition of 95% formamide which occurs when the solution is heated and results in release of ammonia. Second, samples recovered from streptavidin magnetic particles are found to be contaminated with protein derived from streptavidin. Release of immobilized primer extension products results from the denaturation of the streptavidin that is covalently linked to the magnetic particle. Streptavidin is a multi-subunit protein with a high isoelectric point. Denaturation of immobilized streptavidin is always accompanied by the concomitant release of those protein subunits that are not covalently linked to the magnetic particles. This contaminating protein acts in a manner somewhat analogous to template DNA, as a consequence of its anionic character and high molecular weight. Finally, dye-labeled fluorescent dideoxynucleotide terminators and, in particular, the recently developed dye-labeled terminators having two fluorescent labels configured as energy transfer pairs (ABI PRISM BigDye™ Terminators from PE Biosystems and DYEnamic E™ Terminators from Amersham Pharmacia) have been found to bind nonspecifically to streptavidin magnetic particles, and to be released into the formamide solution upon denaturation of streptavidin. Thus, the nonspecifically bound terminators can accompany the “purified” primer extension products and adversely affect their analysis.

The second approach to template DNA removal utilizes a multi-step methodology involving: (1) Ultrafiltration to remove template DNA; (2) Vacuum concentration to reduce sample volume; (3) Size exclusion chromatography (two sequential gel filtration columns) to reduce the ionic strength; and (4) Vacuum concentration to reduce sample volume prior to analysis (Ruiz-Martinez et al., Anal. Chem. 1998, 70, 1516-1527, and Salas-Solano et al., Anal. Chem. 1998, 70, 1528-1535). Although this approach affords excellent samples for CE analysis, it is generally complex, costly, time consuming and unsuitable for automation in a high throughput environment. In fact, as compared to the throughput potential of multi-column capillary electrophoresis DNA sequencers, the aforementioned methodology would constitute the rate-limiting step in a sequencing laboratory.

Thus, none of the methods currently available provide for the quantitative removal of all of the potentially contaminating constituents associated with DNA sequencing reactions. Consequently, a method is needed to circumvent this considerable limitation if the extraordinary potential of capillary electrophoresis for DNA sequencing is to be realized in the not too distant future. The present invention fulfills this and other needs.

### SUMMARY OF THE INVENTION

The present invention provides, in a first embodiment, a composition that includes a complexing agent that can bind to a string of arylboronic acid moieties. In the compositions of the invention, the complexing agent is bound to a solid support, and a string of arylboronic acid moieties is complexed to the complexing agent. The string of arylboronic acid moieties typically is covalently attached to a nucleotide or nucleoside that is generally included in an oligonucleotide or polynucleotide. In presently preferred embodiments, the oligonucleotide is a primer that is enzymatically extended to add additional nucleotides prior to being complexed to the solid support. Generally, the primer is hybridized to a template nucleic acid prior to the primer extension reaction. The extended primer can be the product of any one of many types of primer extension reaction known to those of skill in the art, including, for example, cycle sequencing reactions, polymerase chain reactions, ligase chain reactions, cDNA synthesis reactions and RACE reactions.

Also provided by the invention are methods for purifying a primer extension product. These methods involve:

(a) extending a primer that comprises a string of arylboronic acid moieties using a primer extension reaction to form primer extension products;

(b) contacting the primer extension products of (a) with a solid support having attached thereto an arylboronic acid complexing moiety, to form a complex comprising the primer extension products and the solid support; and

(c) separating the complex of (b) from the liquid phase of the primer extension reaction.

The primer is, in typical embodiments, annealed to a template prior to the primer extension reaction. If desired, the primer extension products can be released from the nucleic acid template by denaturation prior to contacting the primer extension products with the solid support. In a presently preferred embodiment, the complex is washed to remove any uncomplexed reactants after separating the complex from the liquid phase of the primer extension reaction.

The primer extension products then can be disassociated from the complex to obtain the purified primer extension products. The dissociation is preferably effected by elevating the temperature of a liquid that contains the complex. In presently preferred embodiments, the liquid has an ionic strength of between about zero and about 10 mM; water is a preferred liquid. When dissociation is performed using a low ionic strength liquid, the primer extension products can be injected directly onto a capillary electrophoresis column without desalting or concentrating the primer extension products. Competitive displacement, either alone or in combination with temperature elevation, can also be used to dissociate the primer extension products.

In another embodiment, the invention provides methods for isolating a nucleic acid. The methods involve:

(a) contacting a sample comprising the nucleic acid with a probe that comprises a string of arylboronic acid moieties and can hybridize to the nucleic acid, to form a nucleic acid hybrid;

(b) contacting the nucleic acid hybrid of (a) with a solid support having attached thereto a arylboronic acid complexing moiety to form a complex comprising the nucleic acid hybrid and the solid support; and

(c) separating the complex of (b) from the sample.

Also provided by the invention are methods for purifying a nucleic acid sequencing reaction product. The methods involve:

(a) hybridizing a primer comprising a string of arylboronic acid moieties to a nucleic acid template to form a template-primer hybrid;

(b) extending the primer by contacting the hybrid with a polymerase in a reaction mixture comprising deoxynucleotides and dideoxynucleotides to form primer extension products;

(c) contacting the primer extension products of (b) with a solid support having attached thereto a arylboronic acid complexing moiety to form a complex comprising the primer extension products and the solid support; and

(d) separating the complex of (c) from the reaction mixture.

### BRIEF DESCRIPTION OF THE DRAWINGS

The file of this patent contains at least one drawing executed in color. Copies of this patent with color drawing(s) will be provided by the Patent and Trademark Office upon request and payment of the necessary fee.

FIG. 1 is a schematic representation of the method provided by the invention for purifying primer extension products. The primers used in the reactions have a phenylboronic acid moiety at the 5′ terminus. After primer extension, the reaction products are purified by complexation with a solid phase support to which is attached phenylboronic acid complexing moieties. The solid supports are washed, the reaction products are released (e.g., by heating), and the products are analyzed by, for example, slab gel or capillary electrophoresis.

FIG. 2 summarizes the cycle sequencing methodology from which the invention can be used to purify the primer extension products. A sequencing ladder is generated by repetition of several cycles in which a primer is first annealed to template DNA that provides a hybrid suitable for subsequent extension of the primer by the action of a thermal stable DNA polymerase in the presence of deoxynucleotide triphosphates. Each of the primer extension products is eventually terminated by incorporation of dideoxynucleotide triphosphate terminator.

FIG. 3 illustrates the cycle sequencing methodology while emphasizing that a dye-labeled dideoxynucleotide triphosphate terminator can be substituted for an unlabeled terminator, thereby generating a sequencing ladder suitable for detection in an automated DNA sequencer having fluorescence detection capabilities.

FIG. 4 summarizes the method described in FIG. 1. The various steps associated with the method are illustrated using as an example magnetic particles as the solid supports in a multiwell plate format.

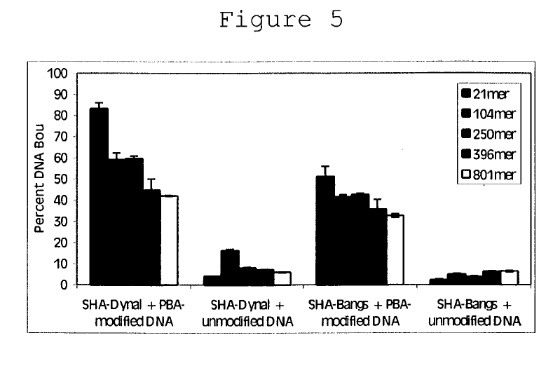

FIG. 5 is a graph illustrating the efficiency and specificity of the capture of a PBA₄-modified oligonucleotide (21 base pairs) and PCR products that are between 104 and 801 base pairs in length on two different SHA-modified magnetic particles.

FIG. 6 is an automated sequencing trace obtained on an ABI PRISM® 373 sequencer utilizing PBA₄-modified cycle sequencing primer extension products in conjunction with ABI PRISM® Big Dye™ terminators.

FIG. 7 illustrates an automated sequencing trace obtained on an Amersham Pharmacia MegaBACE 1000 DNA Sequencing System that employs capillary electrophoresis. The trace resulted from analysis of a 250 base pair PCR product derived from the pUC 18 plasmid, wherein the primer extension products were prepared from PBA₄-modified primer.

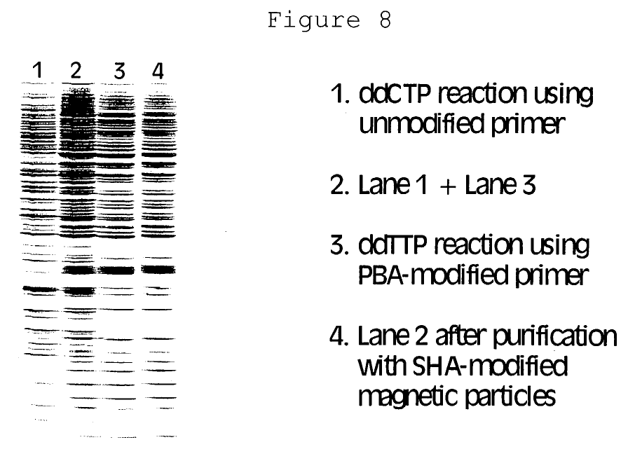

FIG. 8 is a phosphoimage of a ³²P DNA sequencing gel on which is compared the sequence patterns of primer extension reactions prepared using an unmodified primer (Lane 1) or using a PBA₄-modified primer with (Lane 4) or without (Lane 2) purification of the PBA₄-modified primer extension products by capture on SHA-modified magnetic particles. Lane 3 shows the analysis of a mixture of primer extension reactions using the unmodified primer and the modified primer.

FIG. 9 is a phosphoimage of a ³²P DNA sequencing gel which shows a comparison of a polyacrylamide gel electrophoretic analysis of primer extension products produced using an unmodified primer (Lane 1) versus a PBA-modified primer (Lanes 2 (unpurified), 3 and 4 (purified). Purification was by capture on SHA-modified magnetic particles followed by release in water.

FIG. 10 is a graph that illustrates the efficiency of removal of template DNA from PBA₄-modified primer extension products during capture on SHA-modified magnetic particles.

FIGS. 11 and 12 show an automated sequencing trace obtained from an ABI PRISM 310 capillary electrophoresis sequencing apparatus using an unmodified (FIG. 11) and a PBA₄-modified (FIG. 12) primer.

FIG. 13 shows an automated sequencing trace obtained from an ABI PRISM 373 gel electrophoresis sequencing apparatus using a PBA₄-modified primer.

### DETAILED DESCRIPTION OF THE INVENTION AND PREFERRED EMBODIMENTS

Definitions

The following terms and phrases are used herein.

“Nucleoside” and “nucleotide” can refer to either deoxynucleotides or ribonucleotides, and include both naturally occurring molecules and analogs of nucleosides and nucleotides.

“Primer” refers to a single stranded oligonucleotide capable of hybridizing at one or more specific locations or “priming sites” in a template nucleic acid. “Primer extension product” refers to a primer to which one or more naturally occurring or modified nucleotides have been added by template-directed enzymatic addition, typically to the 3′ end of the primer. The process requires hybridization of the primer to the template. A “PBA-primer” is a primer that has one or more pendant phenylboronic acid moieties covalently linked to the 5′ or 3′ end of the primer (most typically the 5′ end). Although some of the discussion herein refers to phenylboronic acids, one can substitute other arylboronic acids for the phenylboronic acids.

A “template” is a single or double stranded nucleic acid that is to be analyzed by means of primer extension reactions. “Primer extension reaction” includes, but is not limited to, a standard Sanger sequencing reaction, a fluorescent terminator sequencing reaction, a polymerase chain reaction, a ligase chain reaction, a cDNA synthesis reaction, or some other template-directed primer extension reaction.

General Overview

The present invention provides methods for the purification of primer extension products. The purified products are free of contaminants, such as polymerase chain reaction and cycle sequencing reaction constituents, and are also free of template DNA. The products are obtained in a form that is optimal for automated DNA sequencing by slab gel or particularly capillary electrophoresis, and for other analytical methods.

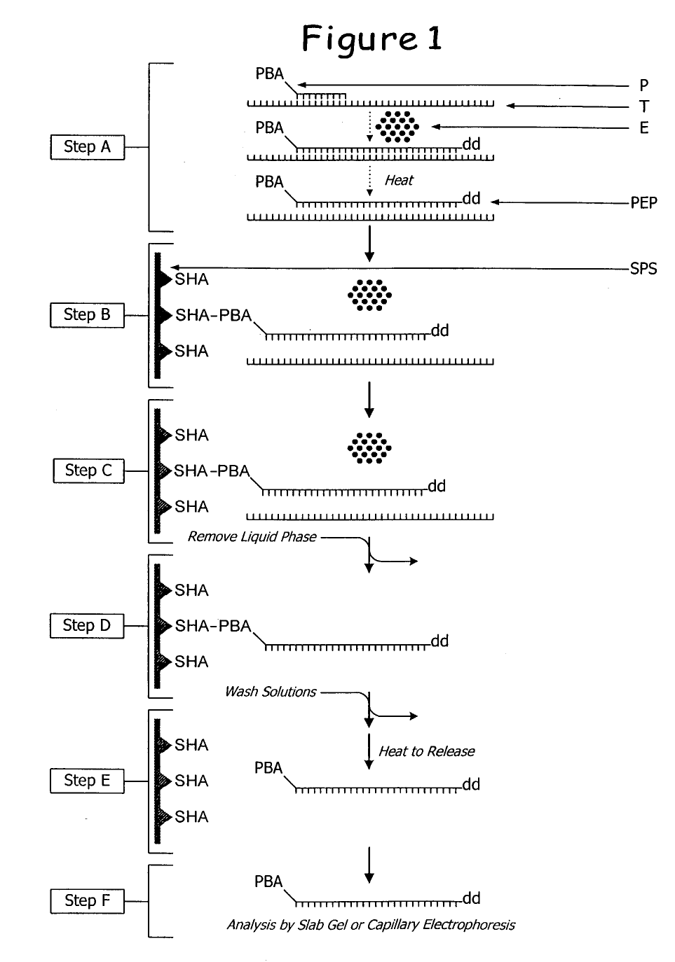

A presently preferred embodiment of the current invention is shown in FIG. 1. In the first step, i.e., Step A, a PBA-primer (P designates the PBA primer, to which is attached one or more phenylboronic acid moieties (PBA)) is annealed to a template nucleic acid (T). The annealed template-primer complex is placed in a reaction mixture that contains a polymerase enzyme (E), dNTPs, ddNTPs, buffer and salts. The polymerase catalyzes the template-directed addition of nucleotides and a dideoxynucleotides to the 3′ end of the primer to create primer extension products (PEP) that terminate in a dideoxynucleotide residue (dd). Typically, the reaction mixture is then heated to denature the primer extension products from the templates, after which the reaction mixture is cooled and the extension reaction is repeated. This cycle can be repeated numerous times as desired. In Step B, the primer extension products are immobilized by attachment to a PBA complexing moiety that is attached to a solid support (SPS). The PBA complexing moiety illustrated in FIG. 1 is salicylhydroxamic acid (SHA). After removal of the liquid phase (i.e., Step C) and one or more washes (i.e., Step D), the primer extension products are released from the solid support by, for example, heating (i.e., Step E). Finally, the purified primer extension products are analyzed by, for example, slab gel or capillary electrophoresis.

The purification methods of the invention provide several advantages over previously known methods for purifying cycle sequencing reaction products. As shown in Table 1, each of ethanol precipitation, spin column purification, and biotin/streptavidin-mediated purification have one or more significant disadvantages. In contrast, the methods of the invention have properties that are optimal for use in capillary electrophoresis.

TABLE 1

| | Optimal for | Phenylboronic | | Spin | |
|---|---|---|---|---|---|
| | Capillary | acid-mediated | Ethanol | Column | Biotin/ |
| | Electrophoresis | Purification | Precipitation | (Size Exclusion) | Streptavidin |
| Buffer, Enzyme, | Yes | Yes | Yes | Yes | Yes |
| Salts & dNTPs | | | | | |
| Removal | | | | | |
| Dye-Labeled | Yes | Yes | No | Yes | No |
| ddNTPs | | | | | |
| Removal | | | | | |
| Template DNA | Yes | Yes | No | No | Yes |
| Removal | | | | | |
| Low Ionic | Yes | Yes | No | Yes | No |
| Strength Product | | | | | |
| Generation of | No | No | No | No | Yes |
| Contaminant(s) | | | | | |
| Ease of | Yes | Yes | No | Yes | Yes |
| Automation | | | (Centrifuge) | (Vacuum) | (Robotic) |
| Relative Cost | Low | Low | Low | Moderate | High |

Primer Extension Reactions

The purification methods of the invention are useful for purifying a wide variety of products that are obtained by polymerase-mediated, template-directed extension of oligonucleotide primers. These reactions are often used in the characterization of nucleic acids, including DNA and RNA. The purification methods can be used, for example, to purify the products of polymerase chain reaction, ligase chain reaction, and other amplification methods that employ primer extension and/or ligation. Primer extension products from analysis of RNA ends can also be purified, as can the products of 5′ and 3′ RACE. cDNA strands can also be purified using the methods of the invention if a PBA-primer is used. These and other protocols that involve primer extension are known to those of skill in the art. Examples of these techniques are found in Berger and Kimmel, Guide to Molecular Cloning Techniques, Methods in Enzymology 152 Academic Press, Inc., San Diego, Calif. (Berger); Sambrook et al. (1989) Molecular Cloning—A Laboratory Manual (2nd ed.) Vol. 1-3, Cold Spring Harbor Laboratory, Cold Spring Harbor Press, NY, (Sambrook et al.); Current Protocols in Molecular Biology, F. M. Ausubel et al., eds., Current Protocols, a joint venture between Greene Publishing Associates, Inc. and John Wiley & Sons, Inc., (1994 Supplement) (Ausubel); Cashion et al., U.S. Pat. No. 5,017,478; and Carr, European Patent No. 0,246,864. Examples of techniques sufficient to direct persons of skill through in vitro amplification methods are found in Berger, Sambrook, and Ausubel, as well as Mullis et al., (1987) U.S. Pat. No. 4,683,202; PCR Protocols A Guide to Methods and Applications (Innis et al. eds) Academic Press Inc. San Diego, Calif. (1990) (Innis); Arnheim & Levinson (Oct. 1, 1990) C&EN 36-47; The Journal Of NIH Research (1991) 3: 81-94; (Kwoh et al. (1989) Proc. Natl. Acad. Sci. USA 86: 1173; Guatelli et al. (1990) Proc. Natl. Acad. Sci. USA 87, 1874; Lomell et al. (1989) J. Clin. Chem., 35: 1826; Landegren et al., (1988) Science, 241: 1077-1080; Van Brunt (1990) Biotechnology, 8: 291-294; Wu and Wallace, (1989) Gene, 4: 560; and Barringer et al. (1990) Gene, 89:117.

Importantly, the PBA moiety attached to the primer does not affect the ability of a variety of enzymes to catalyze primer extension. For example, reverse transcriptase, Taq polymerase, and other DNA polymerases are not impeded by the presence of a PBA moiety at one end of the primer.

The purification methods of the invention are particularly useful where a very clean primer extension product preparation is required. DNA sequencing, in particular where capillary electrophoresis is used, provides an illustrative example of an analytical method for which the methods of the invention can solve major drawbacks that have prevented capillary electrophoresis-mediated DNA sequencing from reaching its full potential.

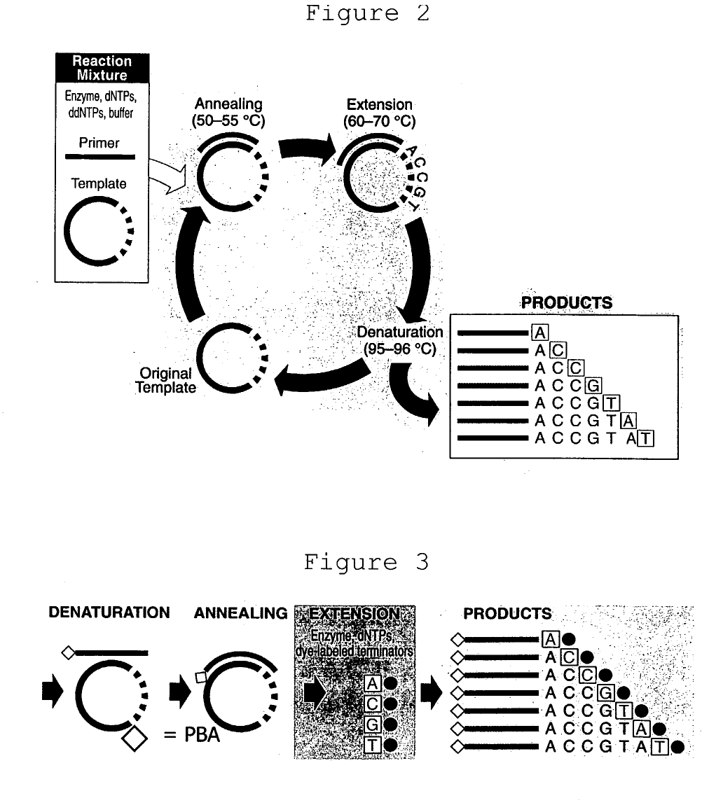

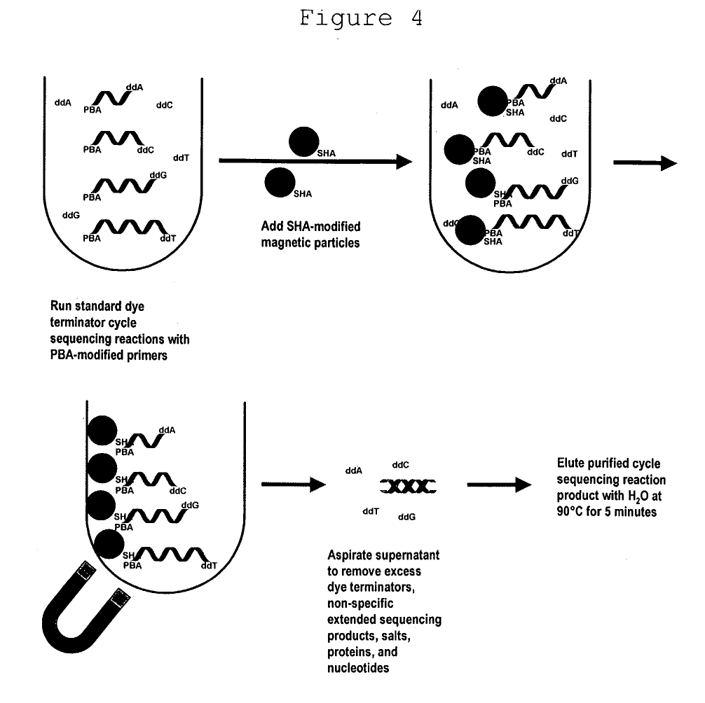

In these methods, a cycle sequencing reaction is carried out as summarized in FIG. 2. In a typical embodiment, a PBA-attached primer is allowed to hybridize to the template DNA at a suitable annealing temperature, which is typically between about 50° and about 55° C., in preparation for primer extension. The polymerase, deoxynucleotides (dNTPs), dideoxynucleotide terminators and other necessary reactants are added to the annealed template-primer complex. The temperature is then raised to an appropriate temperature for the particular polymerase, which is generally between about 60° and about 70° C. for a thermostable polymerase or between about room temperature and about 37° C. for a non-thermostable polymerase, to facilitate template-directed primer extension. Finally, the hybrids formed between the extended primers and the template DNA are denatured, e.g., by heating to a temperature of from about 95° to about 99° C., or other suitable method, thereby effecting release of the terminated primer extension products and liberating the template DNA prior to initiating a second cycle of primer extension. Routinely, this cycle is repeated from about 10 to 25 times. In presently preferred embodiments, the primer extension products produced in the aforementioned cycle contain a dye-labeled dideoxynucleotide terminator and utilize a PBA-modified primer, as illustrated in FIG. 3.

Synthesis of Arylboronic Acid-Linked Primers

The compositions and purification methods of the invention make use of oligonucleotide primers to which are attached one or more arylboronic acid moieties, such as, for example, phenylboronic acid moieties. Generally, a string of two or more arylboronic acid moieties are employed. In a preferred embodiment, the string comprises between about 2 and about 10 arylboronic acids, and in a most preferred embodiment, the string comprises about 4 to about 6 arylboronic acid moieties.

In presently preferred embodiments, the arylboronic acid moieties, e.g., phenylboronic acid (PBA) moieties, are attached to the 5′ end of the oligonucleotide primers. The PBA-oligonucleotides can be prepared from phenylboronic acid that contains phosphoramidite reagents. Suitable arylboronic acid moieties and methods are described in copending, commonly assigned U.S. patent application Ser. No. 09/272,978, titled “Boronic Acid Containing Phosphoramidite Reagents and Polynucleotides”, filed Mar. 19, 1999, and Ser. No. 09/272,834, titled “Boronic Acid Containing Oligonucleotides and Polynucleotides”, filed Mar. 19, 1999, both of which are incorporated herein by reference.

Purification of Primer Extension Products

Upon completion of the primer extension reactions, the extended PBA-primer products are purified by allowing the PBA to form a complex with an arylboronic acid complexing moiety that is attached to a solid support. The solid support is then separated from the unbound components of the reaction mixture.

Prior to, or simultaneously with, incubating the reaction mixture with the solid phase support, it is often beneficial to first separate the template DNA or RNA from the primer extension products, thereby removing a possible source of interference with respect to efficient complexation and analysis of the primer extension products. Methods of denaturing nucleic acids are well known to those of skill in the art. For example, one can heat the reaction mixture to a temperature sufficient to denature the template from the primer extension products. Typically, the reaction mixture is heated to temperature of between about 95° and about 99° C. Other methods of denaturation are known to those of skill in the art.

In some embodiments, however, the nucleic acid is not denatured from the primer prior to the purification of the complexes. For example, in some methods of the invention, the PBA-primer is used to purify a target nucleic acid to which the primer hybridizes. These embodiments can involve primer extension or ligation, or can be performed in the absence of any enzymatic reaction. Upon hybridization of the target nucleic acid to the primer, the PBA-primer-target nucleic acid hybrid is purified by contact with the arylboronic acid complexing moiety without first denaturing the target nucleic acid from the primer. After purification of the resulting complex, the complex can be washed, if desired. The target nucleic acid can then be released from the primer by denaturation.

Following the denaturation step, if performed, the solid supports, which have attached thereto arylboronic acid (e.g., phenylboronic acid) complexing moieties, are placed in the reaction mixture and incubated to effect complexation of the primer extension products having pendant phenylboronic acid moieties to the solid phase support. Preferred phenylboronic acid complexing moieties include, but are not limited to, those derived from salicylhydroxamic acid and 2,6-dihydroxybenzohydroxamic acid. Phenylboronic acid reagents, phenylboronic acid complexing reagents, their conjugates and bioconjugates, as well as methods for their preparation and use are the subject of U.S. Pat. Nos. 5,594,111, 5,623,055, 5,668,258, 5,648,470, 5,594,151, 5,668,257, 5,677,431, 5,688,928, 5,744,627, 5,777,148, 5,831,045, 5,831,046, 5,837,878, 5,847,192, 5,852,178, 5,859,210, 5,869,623, 5,872,224, 5,876,938 and 5,988,297, the teachings of which are incorporated herein by reference.

Suitable solid supports include, but are not limited to, glasses, plastics, polymers, metals, metalloids, ceramics, organics, etc. Suitable solid supports can be flat or planar, or can have substantially different conformations. For example, the supports can exist as particles, beads, strands, precipitates, gels, sheets, tubing, spheres, containers, capillaries, pads, slices, films, plates, slides, etc. Magnetic beads or particles, such as magnetic latex beads and iron oxide particles, are examples of solid substrates that can be used in the methods of the invention. Magnetic particles are described in, for example, U.S. Pat. No. 4,672,040, and are commercially available from, for example, PerSeptive Biosystems, Inc. (Framingham Mass.), Ciba Corning (Medfield Mass.), Bangs Laboratories (Carmel Ind.), and BioQuest, Inc. (Atkinson N.H.). Preferred solid phase supports include, but are not limited to, magnetic beads and particles, chromatographic media and membranes, including membranes comprised of entrapped particulate matter. The separations can be conducted in batch mode, or by passing the solutions through columns that contain the solid support.

The incubation of the reaction mixture with the complexing moieties is generally carried out for at least about 5 min, more preferably at least about 10 min, and most preferably about 15 minutes or more, preferably at room temperature. The incubation step is typically less than about 60 minutes, more preferably is less than about 30 minutes, and most preferably is about 15 minutes.

Once the primer extension products having the attached string of phenylboronic acid moieties have undergone complexation with the solid phase support to which is attached complexing moieties that bind to the phenylboronic acid string, the constituents of the primer extension reaction (e.g., cycle sequencing reaction) that are not complexed to the solid phase support (e.g., template DNA, enzyme, dNTPs, ddNTPs, buffer and salts) are typically removed by washing the solid phase support with one or more wash solutions. The wash solutions can contain reagents, such as detergents or alcohol, that are intended to optimize removal of reactants and other materials that are nonspecifically bound to the solid phase support. Since the next step of the invention involves dissociation of the complexed primer extension products, which preferably is effected by an increase in temperature, the final wash solution will determine the composition of the liquid phase into which the primer extension products are released. Where the purified nucleic acids are to be analyzed by capillary electrophoresis, for example, the final wash solution is preferably water or another solution of low ionic strength.

After the washing steps, the complexed primer extension products are generally dissociated from the solid support-bound complexing moieties. Typically, the dissociation is effected by an increase in temperature. In a presently preferred embodiment, the temperature is increased from room temperature to a temperature that is between about 75° and about 96° C., for a period of time of between about 5 minutes and about 15 minutes. The dissociation is preferably carried out in a low ionic strength solution. Preferably, the ionic strength is about 10 mM or less, more preferably the ionic strength is about 1 mM or less. In presently preferred embodiments, the ionic strength is about zero. For example, water, e.g., double distilled water (ddH₂O), is a preferred dissociation liquid. In this instance, dissociation is thought to result from the mutual repulsion (ion-ion repulsion) which occurs between the surface of the anionic salicylhydroxamate or other arylboronic acid complexing moiety and the anionic primer extension products upon removal of substantially all of the counter ions by washing with water (e.g., ddH₂O) or other low ionic strength solution. The energetics of the repulsive interaction are thought to overcome the energetics of the PBA-SHA complex at elevated temperature, thereby facilitating the hydrolysis of the PBA-SHA complex with the concomitant release of immobilized primer extension products into water or other low ionic strength solution. Although the mechanism of this elution scheme has not been thoroughly elucidated, it provides a clearly attractive alternative to competitive displacement of complexed primer extension products because the primer extension products are removed under conditions which are optimum for electrokinetic injection into automated capillary electrophoresis systems for DNA sequencing.

The efficiency of dissociation can be optionally increased by competitive displacement of the complexed primer extension products by addition of an excess of free arylboronic acid, either alone or in conjunction with the temperature elevation. Arylboronic acids useful for this purpose include, but are not limited to, phenylboronic acid, 4-carboxyphenylboronic acid, 3,5-bis-(dihydroxyboryl)benzoic acid, 4-hydroxy-4,3-boroxaroisoquinoline, 1-hydroxy-1H-2,4,1-benzoxazaborine, 1-hydroxy-3-methyl-1H-2,4,1-benzoxazaborine, and 1-hydroxy-3-trifluoro-methyl-1H-2,4,1-benzoxazaborine. Competitive displacement reagents are generally employed in a concentration range of from about 0.1 millimolar to 10 millimolar.

Unlike analogous methodologies that employ the biotin-avidin system, dissociation of primer extension products according to the methods of the invention does not require the use of denaturing reagents such as formamide, guanidine hydrochloride or urea. In the methods described herein, the purified primer extension products can be recovered in low ionic strength solution, which is advantageous for subsequent analysis by capillary electrophoresis systems for DNA sequencing. The primer extension products obtained using the methods of the invention can be injected directly onto a capillary electrophoresis column without steps such as the desalting or concentrating of the extension products.

Finally, the purified primer extension products, which are free of all other constituents of the extension reaction (e.g., cycle sequencing reaction), can be subjected to analysis by slab gel or preferably by capillary electrophoresis. In most instances, the samples can be injected directly into capillary electrophoresis systems without further processing. Methods for DNA sequencing by capillary electrophoresis are known in the art (see, e.g., Dovichi (1997) Electrophoresis 18: 2393-2399; Kheterpal and Mathies (1999) Anal. Chem. 71: 31A-37A).

Nucleic acids that are purified using the methods of the invention are obtained in a form that is suitable for further enzymatic reactions or other analytical techniques. For example, an RNA that is obtained by hybridization to the PBA-primer and subsequent purification can be subjected to reverse transcription to synthesize a cDNA. Similarly, a cDNA strand that is synthesized using a PBA-primer can be purified according to the methods of the invention, after which a second cDNA strand is synthesized.

### EXAMPLES

The following examples are offered to illustrate, but not to limit the present invention.

### Example 1

### Automated Solid Phase Synthesis and Chromatographic Purification of PBA-Modified Primers for use in PCR and Cycle-Sequencing Reactions

Oligodeoxyribonucleotides were synthesized on a 1 μmole scale using standard automated phosphoramidite chemistry on a Model 394 DNA Synthesizer (Perkin Elmer) in conjunction with the use of UltraFast DNA Synthesis Reagents (Glen Research) in the Trityl ON mode. The completed oligodeoxyribonucleotide was retained on the support. An appropriate quantity of the desired protected PBA-containing phosphoramidite reagent was dissolved either in anhydrous acetonitrile for 1-O-(4,4′-dimethoxytrityl)-8-N-[4-dihydroxyboryl-(benzopinacol cyclic ester) benzoyl)]amino-1,3-octanediol 3-O-(2-cyanoethyl)-N,N-diisopropylamino phosphoramidite and 1-O-(4,4′-dimethoxytrityl)-3-N-[(4-dihydroxyboryl(benzopinacol cyclic ester)benzoyl)-β-alanyl)]amino-1,2-propanediol 3-O-(2-cyanoethyl)-N,N-diisopropylamino phosphoramidite, or in 75:25 (v/v) anhydrous acetonitrile:anhydrous tetrahydrofuran for 1-O-(4,4′-dimethoxytrityl)-2-N-[(4-dihydroxyboryl-(benzopinacol cyclic ester)benzoyl)-β-alanyl)]serinol 3-O-(2-cyanoethyl)-N,N-diisopropylamino phosphoramidite, to give a final concentration of 0.1 M. This solution was placed on the DNA synthesizer in one of the extra phosphoramidite bottle positions. Four (4) PBA moieties were then added onto the 5′-end of the oligodeoxyribonucleotide using a modification of the standard coupling cycle in which the “wait time” for the coupling reaction had been extended to fifteen minutes. Again, the synthesis was carried out in the Trityl ON mode. Coupling yields for the addition of the PBA amidites to the oligodeoxyribonucleotide were estimated to be>95% from the collected trityl solutions of each cycle and from subsequent analytical high performance liquid chromatography (HPLC).

The completed tritylated, PBA-modified oligodeoxyribonucleotide was then cleaved from the support with concentrated ammonium hydroxide on the instrument according to the manufacturer's protocol. The protecting groups on the nucleic acid bases and the boronic acids were simultaneously removed by heating the ammonium hydroxide solution in a heating block at 60° C. for one hour. This solution was then cooled to 4° C. in a refrigerator and concentrated to about 1 mL in a SpeedVac vacuum concentrator (Savant Instruments). The solution containing the crude PBA₄-modified oligodeoxyribonucleotide was stored at 4° C. until purification by high performance liquid chromatography.

Crude tritylated, PBA₄-modified oligodeoxyribonucleotides were purified by reverse phase HPLC using modifications of methods commonly used to purify synthetic oligodeoxyribonucleotides. However, the C18 and C8 phases commonly used to purify tritylated unmodified oligodeoxyribonucleotides and labeled oligodeoxyribonucleotides performed poorly with the tritylated, PBA₄-modified oligodeoxyribonucleotides. Peaks associated with the desired products were very broad, tailed badly, and as such were poorly resolved from impurities. It was found that C4 phases performed better and gave satisfactory results.

An aliquot (10-100 μL) of the above solution of crude tritylated, PBA₄-modified oligodeoxyribonucleotides was injected onto a 4.6 mm×150 mm C4 column (Inertsil 5 μm, MetaChem Technologies) coupled to a Hewlett Packard Series 1050 liquid chromatograph. A linear gradient comprised of acetonitrile (Component B) in 0.1 M triethylammonium acetate, pH 6.5 (Component A), was used to develop the chromatogram. The gradient was as follows: 95:5 (v/v) A:B to 65:35 (v/v) A:B over 21 minutes, then to 10:90 (v/v) A:B over 3 minutes. The flow rate was 1.0 mL/minute, and UV detection at 280 nm was used to observe the separation. The product oligodeoxyribonucleotides eluted from the column at 18-22 minutes. The product was collected and evaporated to dryness in the SpeedVac to afford an oily pellet. The pellet was dissolved in 1 mL of 80:20 (v/v) glacial acetic acid:water and allowed to sit at room temperature for one hour to remove the trityl group. The solution was again evaporated to dryness in the SpeedVac to afford an oily pellet. The pellet was dissolved in 0.5 mL of water and stored frozen. A ten microliter (10 μL) aliquot was analyzed by HPLC using the above column and gradient. Purities of PBA-modified oligodeoxyribonucleotides obtained by this procedure were generally >90%.

### Example 2

### PBA-Modified Primers for the Polymerase Chain Reaction

This example demonstrates that PBA-primers are functional in a polymerase chain reaction. A region of Lambda DNA (801 base pairs) was amplified by the polymerase chain reaction (PCR). The PCR reaction contained 200 μM DATP, dCTP, dGTP and dTTP in addition to PBA-modified oligonucleotide forward primer and unmodified oligonucleotide reverse primer, each at 1 μM in 1× Assay Buffer A (FisherBiotech), 0.1 μg Lambda DNA, and 5 Units of Thermus aquaticus (Taq) DNA polymerase (FisherBiotech). Using a GeneAmp PCR System 9700 Thermal Cycler (Perkin Elmer), the reaction mixture was denatured at 92° C. for one minute and amplified by 35 cycles of PCR at 95° C. for 10 seconds, 62° C. for 20 seconds, and 72° C. for 30 seconds, with a final extension at 72° C. for 5 minutes. The reaction produced 50-100 ng of amplified product (801 base pairs), which exhibited retarded mobility relative to unmodified PCR product during electrophoresis on 1% agarose gels in 50 mM Tris, 100 mM borate, 2 mM EDTA buffer, pH 8.3.

### Example 3

### Preparation of SHA-Magnetic Particles

Ten milliliters (10 mL) of unmodified M280 or M450 magnetic particles (Dynal) were gradually dehydrated into acetonitrile, and converted to aldehyde modified beads by reaction with oxalyl chloride, N,N-dimethylsulfoxide and triethylamine in dichloromethane at −78° C. The resulting aldehyde bearing beads were gradually re-hydrated and suspended in 5 mL of 0.1 M sodium acetate, pH 5.5. The aldehyde groups were coupled with SHA-X-Hydrazide (N-[(4-(N-hydroxycarbamoyl)-3-hydroxyphenyl)methyl]-N′-aminopentane-1,5-diamide) or bis-SHA-Y-Hydrazide (N,N-bis({N-[(4-(N-hydroxycarbamoyl)-3-hydroxyphenyl) methyl] carbamoyl}-methyl)-N′-aminopentane-1,5-diamide) by adding 10-15 milligrams dissolved in 200 μL N,N-dimethylformamide, and rotating the coupling reaction over night at room temperature. The beads were then washed extensively with water and stored in 5 mL of 20% ethanol at 4° C.

Alternatively, 1.5 mL (settled beads) of amine-modified magnetic particles (Bang's Laboratories) were diluted to 15 mL with 0.1 M NaHCO₃. The amine groups were coupled with SA(OCH₂CN)—X—NHS (2,5-dioxopyrrolidinyl 4-[N-({4-[(cyanomethyl)oxycarbonyl]-3-hydroxy-phenyl}methyl)carbamoyl]butanoate) or bis-SA(OCH₂CN)—Y—NHS (2,5-dioxopyrrolidinyl 4-(N,N-bis {[N-({4-[(cyanomethyl) oxycarbonyl]-3-hydroxyphenyl} methyl) carbamoyl]methyl}-carbamoyl)butanoate) by adding 60-70 milligrams dissolved in 1 mL N,N-dimethylformamide, and rotating the coupling reaction over night at room temperature. The beads were then washed extensively with water. The cyanomethyl ester was converted to a hydroxamic acid by adding 20 mL of 1 M NH₂OH, 0.1 M NaHCO₃ (pH 10) to the magnetic particles, and rotated over night at room temperature. The particles were washed extensively with water and stored as a 10% slurry in 20% ethanol at 4° C.

### Example 4

### Efficiency of Capture of PBA-modified PCR Product Using SHA-Modified Magnetic Particles

This Example describes an experiment to determine the time necessary for a PBA-modified PCR product to bind to a complexing agent that binds PBA. A 5′-PBA₄-modified PCR product (801 base pairs) or unmodified PCR product (801 base pairs, 4 pmol), each radiolabeled on the 3′-end using ³²P-cordecypin, was diluted to 40 μL with 3.0 M NaCl, 300 mM sodium citrate, pH 7 (20×SSC), to a final concentration of 100 nM in 10×SSC.

The DNA samples were added to a polypropylene microwell plate containing bis-SHA-modified Dynal or Bang's Laboratories magnetic particles (100 μL of a 10% (v/v) slurry per well) pre-washed three times with 100 μL volumes of water. The particles and the PCR products were mixed by pipetting ten times and then incubated at room temperature for 15, 30, 45 or 60 minutes. At the end of each incubation period, the magnetic particles were captured in the bottom of the wells with a magnetic plate and the supernatant was removed. The magnetic particles were re-suspended in and washed twice with 100 μL volumes of ELISA wash buffer (150 mM NaCl, 20 mM Tris-HCl, and 0.02% (v/v) Tween 20, pH 8). The magnetic particles were captured in the bottom of the wells with a magnetic plate and the supernatant was removed. The magnetic particles were re-suspended in 200 μL ELISA wash, transferred to a scintillation vial and the number of counts per minute (cpms) corresponding to the presence of ³²P were determined.

The SHA-modified magnetic particles incubated for 15, 30, 45 and 60 minutes with PBA₄-modified DNA produced the same number of cpms corresponding to a constant 30% of the total PCR product offered as being bound. This indicates that capturing PBA₄-modified DNA for 15 minutes is as efficient as capturing PBA₄-modified DNA for longer periods of time.

### Example 5

### Efficiency of Capture of Various Lengths of PBA-modified PCR Products on SHA-Modified Magnetic Particles

In this Example, the effect of polynucleotide length on ability to bind to a PBA complexing moiety was examined. The experiment employed 5′-PBA₄-modified PCR products or unmodified PCR products (4 pmol) that were radiolabeled at the 3′-end with ³²P cordecypin. The following lengths were used: a 21 mer oligonucleotide, a 104 base pair PCR product, a 250 base pair PCR product, a 396 base pair PCR product and an 801 base pair PCR product. The polynucleotides were diluted to 40 μL with 3.0 M NaCl, 300 mM sodium citrate, pH 7 (20×SSC), to a final concentration of 100 nM in 10×SSC. The DNA samples were added to a polypropylene multiwell plate containing bis-SHA-modified Dynal or Bang's Laboratories magnetic particles (100 μL of a 10% (v/v) slurry per well) pre-washed three times with 100 μL volumes of water. The particles and the PCR products were mixed by pipetting ten times and then incubated at room temperature for one hour. The magnetic particles were captured in the bottom of the wells with a magnetic plate and the supernatant was removed. The magnetic particles were resuspended in and washed with 2-200 μL volumes of ELISA wash buffer (150 mM NaCl, 20 mM Tris-HCl, and 0.02% (v/v) Tween 20, pH 8). The magnetic particles were again captured in the bottom of the wells with a magnetic plate and the supernatant was removed. The magnetic particles were resuspended in 200 μL of ELISA wash and transferred to a scintillation vial and the number of counts per minute (cpms) determined.

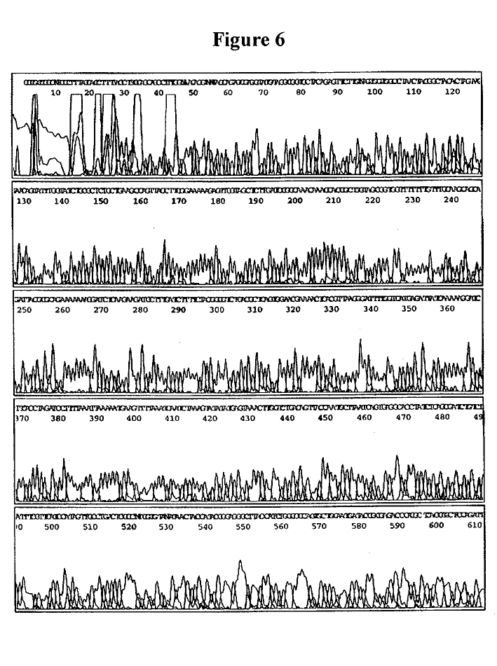

As illustrated in FIG. 5, the SHA-modified magnetic particles treated with unmodified DNA produced cpms corresponding to ≦5% of the total DNA offered as being bound for all DNA lengths, while the SHA-modified magnetic beads treated with PBA-modified PCR product produced cpms corresponding to 30-80% of the total PCR product offered as being bound for all DNA lengths. This indicates the specific immobilization of significant amounts of PBA₄-modified PCR product on the surface of the beads, and that the immobilization is independent of the relative length of the PCR product.

### Example 6

### Specific Release of PBA-modified PCR Product From SHA-Magnetic Particles with PBA-Oxime Reagent

In this example, the release of PBA-modified PCR products from a PBA complexing moiety by competitive binding was analyzed. 5′-PBA₄-modified 396 base pair PCR product (5 pM) was radiolabeled and diluted to 50 μL with 3.0 M NaCl, 300 mM sodium citrate, pH 7 (20×SSC), to a final concentration of 100 nM in 10×SSC. The PCR products were added to a polypropylene multiwell plate containing bis-SHA-modified Dynal or Bang's Laboratories magnetic particles (100 μL of a 10% (v/v) slurry per well) pre-washed three times with 100 μL volumes of water. The particles and the DNA were mixed by pipetting ten times and then incubated at room temperature for 15 minutes. After the incubation period, the magnetic particles were captured in the bottom of the wells with a magnetic plate and the supernatant was removed. The magnetic particles were resuspended in and washed twice with 200 μL volumes of ELISA wash buffer (150 mM NaCl, 20 mM Tris-HCl, and 0.02% (v/v) Tween 20, pH 8). The magnetic particles were again captured in the bottom of the wells with a magnetic plate and the supernatant was removed. The magnetic particles were re-suspended in and washed twice with two times 200 μL volumes of 50 mM Tris, pH 7. The magnetic particles were captured in the bottom of the wells with a magnetic plate and the supernatant was removed.

To effect release, 100 μL of 1 mM PBA-oxime (4-hydroxy-4,3-boroxaroisoquinoline) in 100 mM phosphate buffer, pH 4.5 or 100 μL of 100 mM phosphate buffer, pH 4.5 was added to the samples. The samples were heated at 95° C. for 10 minutes. The magnetic particles were captured in the bottom of the wells with a magnetic plate and the supernatant, containing any released DNA, was removed and transferred to a scintillation vial. The counts per minute (cpms) of the released DNA were determined and compared with the cpms representing the total amount of DNA originally captured on the magnetic particles. Four to ten times more DNA was released from the magnetic particles when PBA-oxime was included in the release solution. This is consistent with the ability of PBA-oxime to specifically elute PBA-modified DNA from bis-SHA modified magnetic particles.

### Example 7

### Compatibility of PBA-modified Primers with Various DNA Polymerases for the Production of Cycle-Sequencing Primer Extension Products

This Example demonstrates that PBA-modified primers are compatible with a variety of DNA polymerases.

AmpliCycle™ Sequencing Kit

A region of Lambda DNA (801 base pairs) was sequenced by DNA Cycle Sequencing using modifications to the AmpliCycle™ Sequencing kit (Perkin Elmer).

The sequencing reactions were carried out by placing, for each sample, 2 μL of a G, A, T, and C Termination Mix (AmpliCycle™ Sequencing kit) into MicroAmp™ Reaction tubes with caps (Perkin Elmer), one tube per Termination Mix. The reaction tubes were maintained on ice. To each tube was added 4 pmol of PBA₄-labeled primer in 1×Cycling Mix (Perkin Elmer), 8 fmol Lambda DNA template (801 base pairs), and 3 μCi of [α-³³P]-dATP (NEN Life Sciences). The volume of each reaction was brought up to a final volume of 8 μL with water. The capped tubes were placed in a GeneAmp™ PCR system 2400 Thermal Cycler (Perkin Elmer) and preheated to 95° C. The reactions were denatured at 95° C. for one minute and extended by 25 thermal cycles at 95° C. for one minute, 68° C. for 30 seconds, and 72° C. for one minute, with a final extension at 72° C. for one minute. After the thermal cycling, 4 μL of stop solution (AmpliCycle™ Sequencing kit) was added to each reaction. The reactions were heated to 95° C. for 5 minutes, placed on ice, and loaded 2 μL per well onto an 8% denaturing acrylamide (Gel-Mix™ 8, Life Technologies) gel in 50 mM Tris, 100 mM borate, 2 mM EDTA, pH 8.3. The gel was subjected to electrophoresis at 2000 V for 1.5 hours.

All four terminated reactions gave readable sequence that matched the known sequence for the 801 base pairs region of Lambda DNA. The PBA₄-modified extension products exhibited retarded mobility relative to unmodified extension products consistent with PBA₄ being present.

SequiTherm EXCEL II DNA Sequencing Kit™

A region of Lambda DNA (801 base pairs) was sequenced by DNA Cycle Sequencing using modifications to the SequiTherm EXCEL II DNA Sequencing kit™ (Epicentre Technologies). For each sample, 2 μL of a G, A, T, and C SequiTherm EXCEL II Termination Mix (SequiTherm EXCEL II DNA Sequencing kit™) were dispensed into MicroAmp Reaction tubes with caps (Perkin Elmer), one tube per Termination Mix. The reaction tubes were maintained on ice. To each tube was added 4 pmol of PBA₄-labeled primer in 1×(SequiTherm EXCEL II Sequencing Buffer), 15 fmol Lambda DNA template (801 base pairs), 0.2 μL, 5U/μL DNA Polymerase and 3 μCi of [α-³³P]-dATP (NEN Life Sciences). The volume of each reaction was brought up to a final volume of 6 μL with water. Capped tubes were placed in a GeneAmp PCR System 2400 Thermal Cycler (Perkin Elmer), preheated to 95° C.

The reactions were denatured at 95° C. for one minute and extended by 25 thermal cycles at 95° C. for one minute, 68° C. for thirty seconds, 72° C. for one minute, with a final extension at 72° C. for one minute. After the thermal cycling, 3 μL of Stop/Loading Buffer (SequiTherm EXCEL II™ Sequencing kit) were added to each reactions. The reactions were heated to 95° C. for 5 minutes, placed on ice and loaded onto an 8% denaturing acrylamide (Gel-Mix™ 8, Life Technologies) gel in 50 mM Tris, 100 mM borate, 2 mM EDTA, pH 8.3. The gel was subjected to electrophoresis at 2000 V for 1.5 hours. All four terminated reactions gave readable sequence that matched the known sequence for the 801 base pairs region of Lambda DNA. The PBA₄-modified extension products exhibited retarded mobility relative to unmodified extension products consistent with PBA₄ being present.

### Example 8

### Compatibility of PBA-Modified Primers with the ABI PRISM BigDye Terminator Cycle Sequencing Ready Reaction Kit™

The experiments described in this Example demonstrate that PBA-modified primers are compatible with the ABI PRISM BigDye Terminator Cycle Sequencing Ready Reaction Kit.™ A region of pUC18 plasmid DNA (1 kilobase) was sequenced by DNA Cycle Sequencing using modifications to the ABI PRISM BigDye Terminator Cycle Sequencing Ready Reaction Kit™ (Perkin Elmer). For each sample, dispensed 8 μL of Terminator Ready Reaction Mix (ABI PRISM BigDye Terminator Cycle Sequencing Ready Reaction Kit™) into MicroAmp™ Reaction tubes with caps (Perkin Elmer). To each tube was added 3.2 pmol of PBA₄-labeled primer and 13 fmol pUC18 PCR template (1 kilobase). The volume of each reaction was brought up to a final volume of 20 μL with water. Capped tubes were placed in a GeneAmp PCR System 9700 Thermal Cycler (Perkin Elmer), preheated to 95° C. The reactions were denatured at 95° C. for five minutes and extended by 25 thermal cycles at 96° C. for ten seconds, 50° C. for five seconds and 60° C. for four minutes.

After the thermal cycling, the reactions were purified away from dye terminators using AGCT Centriflex™ gel filtration cartridges (Edge BioSystems). The cartridges were pre-spun in a centrifuge for 1 minute at 750×g. The reaction was added to the top of the column bed and the cartridge was spun for 1 minute at 750×g. The eluent was collected and dried under vacuum (Savant SpeedVac DNA 110™) at medium temperature for 30 minutes.

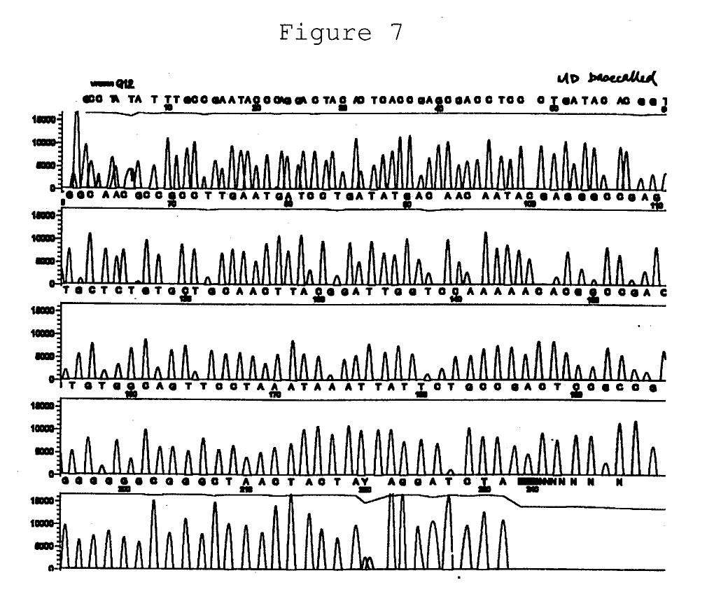

The reactions were re-suspended in 4 μL of 28% deionized formamide, 4 mM EDTA, and 2.8 mg/mL blue dextran in 25 mM Tris, 50 mM borate (pH 8.3). The reactions were heated to 95° C. for 5 minutes and stored at 4° C. Prior to electrophoresis, the reactions were heated a second time to 95° C. for 5 minutes and placed on ice. Two microliter (2 μL) samples were loaded onto a 5% acrylamide (Gel-Mix 8™, Life Technologies) 6 M urea denaturing gel in 100 mM Tris, 90 mM borate, 2 mM EDTA, pH 8.3. The gel was subjected to electrophoresis at 2800 V for 15 hours on an ABI PRISM 373 Automated Sequencer™, and the sequence analyzed using the ABI PRISM DNA Sequencing Analysis Software (version 3.3). As illustrated in FIG. 6, the dye-terminated reactions gave 600 base pairs of readable sequence that matched the known sequence for that region of the pUC 18 plasmid.

### Example 9

### Purification of PBA-Modified Cycle-Sequencing Primer Extension Products on SHA Magnetic Particles

The experiments described in this Example demonstrate the purification of PBA-modified cycle sequencing reaction products using SHA magnetic particles as the PBA binding moieties.

A one kilobase PCR product obtained by amplification of pUC 18 plasmid DNA was sequenced by DNA cycle sequencing using modifications to the ABI PRISM BigDye Terminator Cycle Sequencing Ready Reaction Kit™ (Perkin Elmer). For each sample, 8 μL of Terminator Ready Reaction Mix™ (ABI PRISM BigDye Terminator Cycle Sequencing Ready Reaction Kit) was dispensed into MicroAmp™ Reaction tubes with caps (Perkin Elmer). To each tube was added 3.2 pmol of PBA₄-labeled primer and 13 fmol PCR product template DNA (1 kilobase). The volume of each reaction was brought up to a final volume of 20 μL with water. Capped tubes were placed in a GeneAmp PCR System 9700 Thermal Cycler™ (Perkin Elmer), preheated to 95° C. The reactions were denatured at 95° C. for five minutes and extended by 25 thermal cycles at 96° C. for ten seconds, 50° C. for five seconds and 60° C. for four minutes.

Each of the cycle-sequencing reactions (20 μL per reaction) containing 5′-PBA₄-modified extension products (1 kilobase) was diluted with an equal amount (25 μL) of 3.0 M NaCl, 300 mM sodium citrate, pH 8.5 (final concentration 1.7 M NaCl, 170 mM sodium citrate, pH 8.5). The extension products were added to a polypropylene microwell plate containing bis-SHA-modified Bang's magnetic particles (50 μL of a 10% (v/v) slurry per well) that had been pre-washed three times with 100 μL volumes of water. The particles and the extension products were mixed by pipetting ten times and then incubated at room temperature for fifteen minutes. The magnetic particles were captured in the bottom of the wells with a magnetic plate and the supernatant was removed. The magnetic particles were resuspended in, and washed twice with, 100 μL volumes of ELISA wash buffer (150 mM NaCl, 20 mM Tris-HCl, and 0.02% (v/v) Tween 20, pH 8). The magnetic particles were again captured in the bottom of the wells with a magnetic plate and the supernatant was removed. The magnetic particles were washed twice with 100 μL of 50 mM Tris-HCl, pH 7. The magnetic particles were captured in the bottom of the wells with a magnetic plate and the supernatant was removed.

To effect release, 50 μL of 1 mM PBA-oxime (4-hydroxy-4,3-boroxaroisoquinoline) solution in 100 mM phosphate buffer, pH 4.5 containing 2% N,N-dimethylformamide was added, and the magnetic particles mixed by pipetting ten times. The reactions were incubated at 90° C. for 5 minutes. The magnetic particles were captured to the bottom of the wells with a magnetic plate and the supernatants, containing the released extension reactions, were transferred to clean Eppendorf™ tubes. The reactions were dried under vacuum in a SpeedVac™ vacuum concentrator (Savant Instruments) at medium temperature for 30 minutes, and then re-suspended in 2 μL of 40% deionized formamide, 7 M urea and 8 mg/mL blue dextran. The reactions were stored at 4° C. Prior to electrophoresis, the reactions were heated to 95° C. for 5 minutes and placed on ice. Two microliter samples were loaded onto a 5% acrylamide (Gel-Mix 8; Life Technologies) 6 M urea denaturing gel in 100 mM Tris, 90 mM borate, 2 mM EDTA, pH 8.3. The gel was subjected to electrophoresis at 2800 V for 15 hours on an ABI PRISM 373 Automated Sequencer™(Perkin Elmer) and the sequence analyzed using the ABI PRISM DNA Sequencing Analysis software, version 3.3 (Perkin Elmer). The dye-terminated reactions gave 380 base pairs of readable sequence that matched the known sequence for that of the pUC 18 plasmid.

### Example 10

### Specific Capture of PBA-Modified Cycle-Sequencing Primer Extension Products on SHA-Magnetic Particles

In this Example, the specificity of capture of PBA-modified primer extension products on PBA complexing moieties is demonstrated. The following process consists of sequencing a region of Lambda DNA (801 base pairs) using both PBA-modified and unmodified primers followed by the specific capture and release of only the PBA-modified cycle-sequencing primer extension products and then analyzing those same extension products on a DNA sequencing gel.

The DNA was sequenced by DNA cycle sequencing using modifications to the AmpliCycle Sequencing kit™ (Perkin Elmer). For the unmodified DNA sample, 10 μL of the C Termination Mix (AmpliCycle Sequencing kit™) was dispensed into a MicroAmp™ Reaction tube with a cap (Perkin Elmer). The reaction tube was maintained on ice. To the tube was added 20 pmol of unmodified primer in 1×Cycling mix (Perkin Elmer), 100 fmol Lambda DNA template (801 base pairs) and 19 μCi of α-³³P-dATP. For the PBA₄-modified DNA sample, 10 μL of the T Termination Mix (AmpliCycle Sequencing kit™) was dispensed into a MicroAmp™ Reaction tube with a cap (Perkin Elmer). The reaction tube was maintained on ice. To the tube was added 20 pmol of PBA₄-modified primer in 1×Cycling mix (Perkin Elmer), 100 fmol Lambda DNA template (801 bp) and 19 μCi of α-³³P-dATP. The volume of each reaction was brought up to a final volume of 40 μL with water. The capped tubes were placed in a GeneAmp PCR System 2400 Thermal Cycler™ (Perkin Elmer), preheated to 95° C. The reactions were denatured at 95° C. for one minute and extended by 25 thermal cycles at 95° C. for 30 seconds, 68° C. for 30 seconds, and 72° C. for one minute, with a final extension at 72° C. for one minute.

After the thermal cycling, 9 μL of each reaction was combined and the total reaction mixture diluted to 36 μL with an equal volume (18 μL) of 3.0 M NaCl, 300 mM sodium citrate, pH 8.3 (20×SSC). The DNA samples were added to a polypropylene multiwell plate containing bis-SHA-modified Bang's Laboratories magnetic particles (100 μL of a 10% (v/v) slurry per well) that had been pre-washed three times with 200 μL volumes of water. The particles and the DNA were mixed by pipetting ten times and then incubated at room temperature for 15 minutes. At the end of each incubation period, the magnetic particles were captured in the bottom of the wells with a magnetic plate and the supernatant was removed. The magnetic particles were then resuspended in, and washed twice with, 200 μL volumes of ELISA wash buffer (150 mM NaCl, 20 mM Tris-HCl, and 0.02% (v/v) Tween 20, pH 8). Between washings, the magnetic particles were captured in the bottom of the wells with a magnetic plate and the supernatants were removed. The magnetic particles were resuspended in and washed with two times 200 μL volumes of water. Again, between washings, the magnetic particles were captured in the bottom of the wells with a magnetic plate and the supernatants were removed.

To effect release of the bound extension products, 20 μL of water was added to the magnetic particles and the particles were heated at 90° C. for 5 minutes. The supernatant was transferred to a clean 1.7 mL Eppendorf™ tube, and the reaction volume concentrated, under vacuum, to 6 μL in a SpeedVac™ vacuum concentrator (Savant Instruments). Three microliters (3 μL) of Stop solution (Perkin Elmer Sequencing kit) were added to each reaction. The reactions were heated to 90° C. for 5 minutes, placed on ice and 4.5 μL samples were loaded onto an 8% denaturing acrylamide (Gel-Mix™ 8, Life Technologies) gel in 50 mM Tris, 100 mM borate, 2 mM EDTA, pH 8.3. The gel was subjected to electrophoresis at 2000 V for 1.5 hours. The gel was transferred to a sheet of gel filter paper and dried under vacuum at 80° C. for 2 hours. The gel was analyzed by employing a phosphoimager.

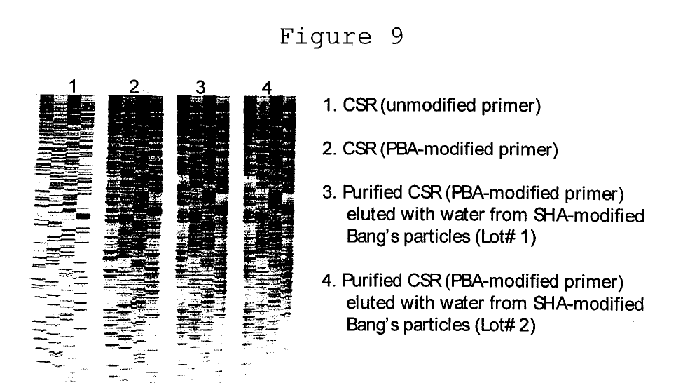

The conclusions are based upon the gel image which is illustrated in FIG. 8. Lanes 1 and 3 contain the cycle sequencing primer extension products synthesized using either an unmodified primer with a dideoxy-C terminator (Lane 1) or a PBA₄-modified primer with a dideoxy-T terminator (Lane 3). The lanes are clearly different. Lane 2 is an equal mixture of the samples which were analyzed independently in Lanes 1 and 3. Lane 4 is identical to Lane 2, except that it was purified using bis-SHA modified magnetic particles as described above. The bands in Lane 4 match those of Lane 3, and demonstrate that the PBA₄-modified cycle sequencing primer extension products were captured and released specifically in the presence of unmodified cycle sequencing primer extension products.

### Example 11

### Specific Removal of Template DNA During Purification of PBA-Modified Cycle-Sequencing Primer Extension Products on SHA-magnetic Particles

This Example demonstrates that template DNA is specifically removed during the purification of PBA-modified cycle sequencing primer extension products on PBA complexing moieties.

A region of Lambda DNA (801 base pairs) was sequenced by DNA cycle sequencing using modifications to the AmpliCycle™ Sequencing kit (Perkin Elmer). For each sample, 5 μL of a G, A, T, and C Termination Mix (AmpliCycle™ Sequencing kit) were dispensed into MicroAmp™ Reaction tubes with caps (Perkin Elmer), one tube per Termination Mix. The reaction tubes were maintained on ice. To each tube was added 10 pmol of PBA₄-labeled primer in 1×Cycling Mix (Perkin Elmer) and 50 fmol ³²P end-labeled Lambda DNA template (801 base pairs). The volume of each reaction was brought up to a final volume of 20 μL with water. The capped tubes were placed in a GeneAmp PCR System 2400 Thermal Cycler™ (Perkin Elmer) and preheated to 95° C. The reactions were denatured at 95° C. for one minute and extended by 25 thermal cycles at 95° C. for one minute, 68° C. for 30 seconds, and 72° C. for one minute, with a final extension at 72° C. for one minute.

After the thermal cycling, 8 μL of each reaction was diluted to 16 μL with an equal volume of 3.0 M NaCl, 300 mM sodium citrate, pH 7 (20×SSC). The DNA samples were added to a polypropylene microwell plate containing bis-SHA-modified Dynal or Bang's Laboratories magnetic particles (50 μL of a 10% (v/v) slurry per well) that had been pre-washed three times with 100 μL volumes of water. The particles and the DNA were mixed by pipetting ten times and then incubated at room temperature for 15 minutes. At the end of each incubation period, the magnetic particles were captured in the bottom of the wells with a magnetic plate, and the supernatant was removed and transferred to a scintillation vial. The magnetic particles were then resuspended in, and washed two times with, 200 μL volumes of ELISA wash buffer (150 mM NaCl, 20 mM Tris-HCl, and 0.02% (v/v) Tween 20, pH 8). Between washings, the magnetic particles were captured in the bottom of the wells with a magnetic plate, and the supernatants were removed and added to the scintillation vial containing the original supernatant. The magnetic particles were resuspended in 200 μL ELISA wash, and transferred to a second scintillation vial. The number of counts per minute (cpms) was determined for all of the scintillation vials containing magnetic particles or supernatants with washes.

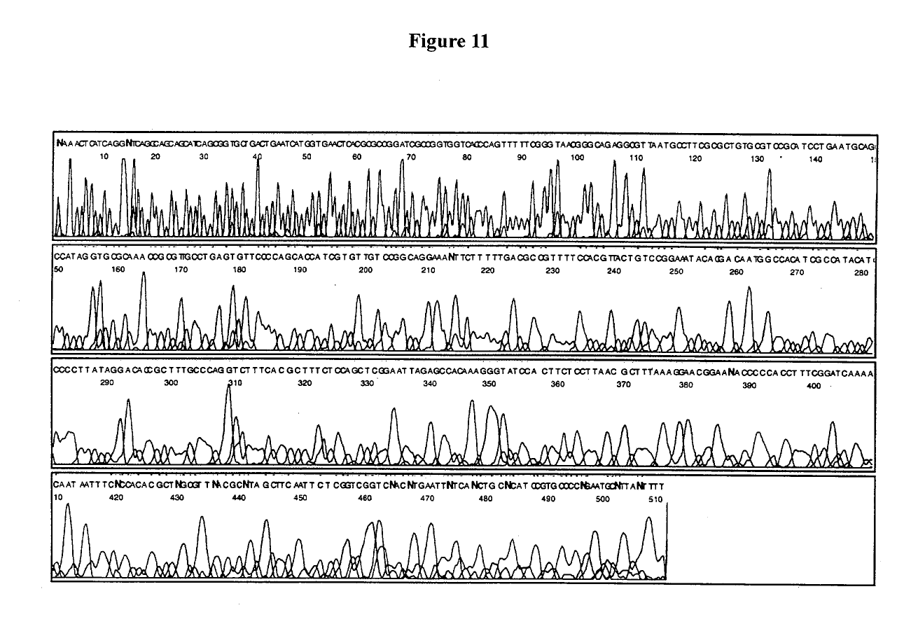

As illustrated in FIG. 10, for all four reactions (A, G, C, and T), the vast majority (93%) of the cpms corresponding to template DNA were found in the scintillation vials containing the supernatant and washes, while only 7% was found to be associated with the magnetic particles. This is consistent with the specific removal of template DNA from PBA-modified cycle-sequencing primer extension products purified on bis-SHA modified magnetic particles.

### Example 12

### Specific Release of PBA-modified Cycle-Sequencing Primer Extension Products From Magnetic Particles with Water

The experiment described in this Example demonstrates that PBA-modified cycle sequencing primer extension reaction products are specifically released from magnetic particle-bound PBA complexing moieties in water. The resulting free products are free of salts and other components that could otherwise interfere with analysis by capillary electrophoresis.

A region of Lambda DNA (801 base pairs) was sequenced by DNA cycle sequencing using modifications to the AmpliCycle™ Sequencing kit (Perkin Elmer). For each sample, 2 μL of a G, A, T, and C Termination Mix were dispensed into MicroAmp™ Reaction tubes with caps (Perkin Elmer), one tube per Termination Mix. The reaction tubes were maintained on ice. To each tube was added 4 pmol of PBA₄-modified primer or unmodified primer in 1×Cycling mix (Perkin Elmer), 20 fmol Lambda DNA template (801 base pairs) and 4 μCi of [α-³³P]-dATP. The volume of each reaction was brought up to a final volume of 8 μL with water. The capped tubes were placed in a GeneAmp PCR System 2400 Thermal Cycler (Perkin Elmer) and preheated to 95° C. The reactions were denatured at 95° C. for one minute and extended by 25 thermal cycles at 95° C. for 30 seconds, 68° C. for 30 seconds, and 72° C. for one minute, with a final extension at 72° C. for one minute.

After the thermal cycling, the reactions were purified on bis-SHA magnetic particles. Eight microliters, (8 μL) of each reaction was diluted to 16 μL with an equal volume (8 μL) of 3.0 M NaCl, 300 mM sodium citrate, pH 8.3 (20×SSC). The DNA samples were added to a polypropylene microwell plate containing bis-SHA-modified Bang's Laboratories magnetic particles (100 μL of a 10% (v/v) slurry per well) that had been pre-washed three times with 200 μL volumes of water. The particles and the DNA were mixed by pipetting ten times and then incubated at room temperature for 15 minutes. At the end of each incubation period, the magnetic particles were captured in the bottom of the wells with a magnetic plate and the supernatant was removed. The magnetic particles were then re-suspended in, and washed twice with, 200 μL volumes of ELISA wash buffer (150 mM NaCl, 20 mM Tris-HCl, and 0.02% (v/v) Tween 20, pH 8). Between washings, the magnetic particles were captured in the bottom of the wells with a magnetic plate and the supernatants were removed. The magnetic particles were resuspended in, and washed twice with, 200 μL volumes of water. Again, between washings, the magnetic particles were captured in the bottom of the wells with a magnetic plate and the supernatants were removed.

To effect release of the bound extension products, 20 μL of water was added to the magnetic particles and the particles were heated at 90° C. for 5 minutes. The supernatant was transferred to a clean 1.7 mL Eppendorf™ tube and the reaction volume concentrated, under vacuum, to 6 μL in a SpeedVac™ vacuum concentrator (Savant Instruments). Three microliters (3 μL) of Stop solution (Sequencing kit, Perkin Elmer) were added to each reaction. The reactions were heated to 90° C. for 5 minutes, placed on ice and 4.5 μL samples were loaded onto an 8% denaturing acrylamide (Gel-Mix 8, Life Technologies) gel in 50 mM Tris, 100 mM borate, 2 mM EDTA, pH 8.3. The gel was subjected to electrophoresis at 2000 V for 1.5 hours. The gel was transferred to a sheet of gel filter paper and dried under vacuum at 80° C. for 2 hours. The gel was exposed using a phosphoimager.

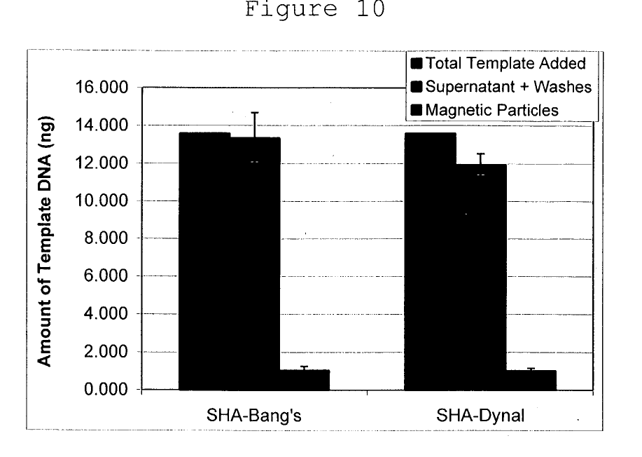

The conclusions are based upon the gel image illustrated in FIG. 9. Lanes 1-4 contain the cycle sequencing primer extension products synthesized using an unmodified DNA primer (T, G, C and A). Lanes 5-8 contain the cycle sequencing primer extension products synthesized using a PBA₄-modified DNA primer (T, G, C and A). There is a slight retardation of mobility of the PBA₄-modified cycle sequencing extension products as compared to the mobility of the unmodified cycle sequencing primer extension products. Lanes 9-12 and Lanes 13-16 are identical to Lanes 5-8, except that they were purified using bis-SHA magnetic particles and released using water as described above. The bands in Lanes 9-12 and Lanes 13-16 match those of lanes 5-8, and demonstrate that the PBA₄-modified cycle sequencing extension products were specifically released from bis-SHA modified magnetic particles using water.

### Example 13

### Purification of PBA-modified Cycle-Sequencing Extension Products on bis-SHA Magnetic Particles and Sequence Analysis on by Gel Electrophoresis and Capillary Electrophoresis

A 790 bp PCR product from lambda DNA was sequenced by DNA Cycle Sequencing using modifications to the ABI PRISM Dye Terminator Cycle Sequencing Core Kit® (Perkin Elmer). For each PBA₄-modified sample, 8 μL of Reaction Premix (Perkin Elmer) was dispensed into MicroAmp® Reaction tubes with caps (Perkin Elmer). To each tube was added 3.2 pmol of PBA₄-labeled primer and 200 fmol PCR product template DNA (790 bp). The volume of each reaction was brought up to a final volume of 20 μL with water. For each unmodified sample, 8 μL of Reaction Premix (Perkin Elmer) was dispensed into MicroAmp® Reaction tubes with caps (Perkin Elmer). To each tube was added 3.2 pmol of unmodified primer and 200 fmol PCR product template DNA (790 bp). The volume of each reaction was brought up to a final volume of 20 μL with water. The capped tubes were placed in a Perkin Elmer GeneAmp® PCR system 9700 thermal cycler and preheated to 95° C. The reactions were denatured at 95° C. for five minutes and extended by 25 thermal cycles at 96° C. for ten seconds, 50° C. for five seconds and 60° C. for four minutes.

Each of the cycle-sequencing reactions (20 μL per reaction) containing 5′-PBA₄-modified extension products (790 bp) was diluted with (25 μL) of 3.0 M NaCl, 300 mM sodium citrate, pH 8.5 (final concentration 1.7 M NaCl, 170 mM sodium citrate, pH 8.5). The extension products were added to a polypropylene microwell plate well containing bis-SHA-modified Bang's magnetic particles (100 μL of a 10% (v/v) slurry per well) that had been pre-washed three times with 200 μL volumes of water. The particles and the extension products were mixed by pipeting ten times and then incubated at room temperature for fifteen minutes. The magnetic particles were captured in the bottom of the wells with a magnetic plate and the supernatant was removed. The magnetic particles were resuspended in, and washed twice with, 200 μL volumes of ELISA wash buffer (150 mM NaCl, 20 mM Tris-HCl, and 0.02% (v/v) Tween 20, pH 8). Again, the magnetic particles were captured in the bottom of the wells with a magnetic plate and the supernatant was removed. The magnetic particles were washed twice with 200 μL volumes of water. The magnetic particles were captured in the bottom of the wells with a magnetic plate and the supernatant was removed.

To effect release, 20 μL of water was added, and the magnetic particles mixed by pipeting ten times. The reactions were incubated at 90° C. for 5 minutes. The magnetic particles were captured to the bottom of the wells with a magnetic plate and the supernatants, containing the released extension reactions, were transferred to clean Eppendorf® tubes. The magnetic particles were rinsed with 20 μL of water and the rinse solutions were added to the supernatants. The reactions were concentrated under vacuum (Savant DNA SpeedVac® DNA 110) to 10 μL using medium temperature for 30 minutes. To each tube added 10 μL of deionized formamide.

Each of the cycle-sequencing reactions (20 μL per reaction) containing unmodified extension products (790 bp) was purified using AGCT Centriflex™ Gel Filtration Cartridges (Edge Biosystems). The cartridges were pre-spun in a centrifuge for one minute at 750×g. The cartridges were washed seven times with 250 μL aliquots of doubly distilled water (ddH₂0), spinning for one minute at 750×g between washes. The cartridges were spun dry for 15 seconds at 750×g. For each reaction, the sample was overlayed on the top of the column bed and spun the cartridge for one minute at 750×g. The eluate was collected and dried under vacuum (Savant DNA SpeedVac® DNA 110) at medium temperature for 30 minutes.

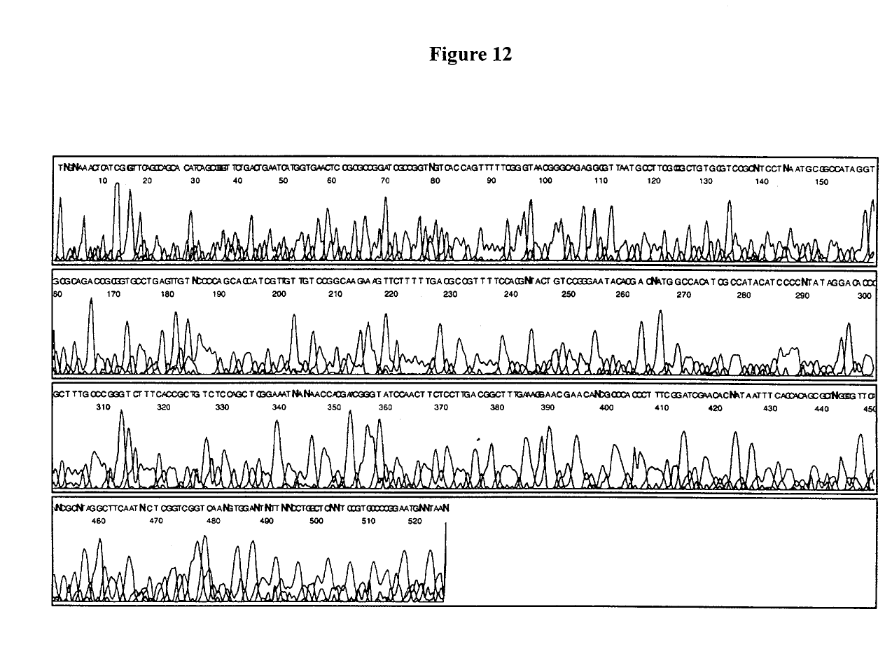

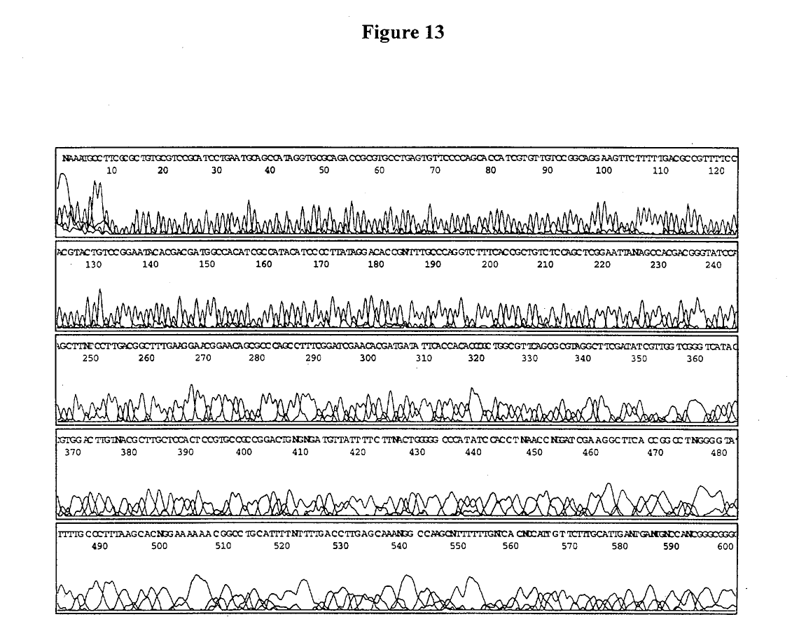

Prior to electrophoresis, the reactions were heated twice to 95° C. for 4 minutes and placed on ice. The reactions were electrokinetically injected at 2 kV for 30 seconds on a 47 cm, POP6 sequencing polymer-containing DNA sequencing capillary (Perkin Elmer) in an ABI PRISM 310 sequencing apparatus, using an unmodified (FIG. 11) or a PBA₄-modified (FIG. 12) primer. The extension products were resolved during electrophoresis at 15V for 45 minutes. Additional aliquots of the samples were analyzed on an ABI PRISM 373 polyacrylamide gel electrophoresis apparatus (FIG. 13) and after purification. As shown in FIGS. 11-13, the PBA₄-modified dye-terminated reactions gave over 400 base pairs of readable sequence that matched the sequence for the unmodified dye-terminated reactions and the known sequence for that region of lambda DNA.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes.

What is claimed is:

1. A composition comprising an arylboronic acid complexing agent bound to a solid support, wherein a string of two or more arylboronic acid moieties are complexed to the complexing agent.

2. The composition of claim 1, wherein the string comprises between 2 and about 10 arylboronic acid moieties.

3. The composition of claim 2, wherein the string comprises between 4 and 6 arylboronic acid moieties.

4. The composition of claim 1, wherein the arylboronic acid moieties are attached to a nucleotide or nucleoside.

5. The composition of claim 4, wherein the nucleotide comprises an oligonucleotide.

6. The composition of claim 5, wherein the oligonucleotide is a primer.

7. The composition of claim 6, wherein the primer is enzymatically extended prior to being bound to the complexing agent.

8. The composition of claim 7, wherein the primer is annealed to a template prior to being enzymatically extended to make an extended primer.

9. The composition of claim 8, wherein the extended primer is a product of a reaction selected from the group consisting of cycle sequencing reaction, polymerase chain reaction, ligase chain reaction, cDNA synthesis reaction and a RACE reaction.

10. A method for purifying a primer extension product, said method comprising:
(a) extending a primer that comprises a string of arylboronic acid moieties using a primer extension reaction to form primer extension products;
(b) contacting the primer extension products of (a) with a solid support having attached thereto an arylboronic acid complexing moiety to form a complex comprising the primer extension products and the solid support; and
(c) separating the complex of (b) from the liquid phase of the primer extension reaction.

11. The method of claim 10, wherein the primer is annealed to a template prior to the primer extension reaction.

12. The method of claim 11 further comprising denaturing the primer extension products from the nucleic acid template prior to the contacting step.

13. The method of claim 10, further comprising
(d) washing the complex to remove any uncomplexed reactants.

14. The method of claim 10, further comprising:
(d) dissociating the primer extension products from the complex.

15. The method of claim 10, further comprising:
(d) dissociating the primer extension products from the complex; and
(e) analyzing the primer extension products.

16. The method of claim 10, further comprising:
(d) washing the complex to remove any uncomplexed reactants;
(e) dissociating the primer extension products from the complex; and
(f) analyzing the primer extension products.

17. The method of claim 14, wherein the dissociation is effected by elevating the temperature of a liquid that comprises the complex.

18. The method of claim 17, wherein the liquid has an ionic strength of between about zero and about 10 mM.

19. The method of claim 18, wherein the primer extension products are injected directly onto a capillary electrophoresis column without desalting or concentrating the primer extension products.

20. The method of claim 18, wherein the liquid has an ionic strength of between about zero and 1 mM.

21. The method of claim 20, wherein the liquid is water.

22. The method of claim 14, wherein the dissociation is effected by competitive displacement.

23. The method of claim 22, wherein the primer extension product is dissociated by competitive displacement using a free arylboronic acid.

24. The method of claim 15, wherein the analysis is by gel electrophoresis or capillary electrophoresis.

25. The method of claim 10, wherein the primer extension reaction is selected from the group consisting of cycle sequencing reactions, polymerase chain reactions, ligase chain reactions, cDNA synthesis reactions and RACE reactions.

26. The method of claim 10, wherein the string of arylboronic acid moieties is attached to the 5′ end of the primer.

27. The method of claim 10, wherein the string of arylboronic acid moieties is attached to the 3′ end of the primer.

28. The method of claim 10, wherein the string of arylboronic acid moieties comprises between 2 and about 10 arylboronic acid moieties.

29. The method of claim 10, wherein the solid support is a member selected from the group consisting of glasses, plastics, polymers, metals, metalloids, chromatography media, ceramics and organics.

30. The method of claim 10, wherein the solid support is a member selected from the group consisting of magnetic beads and magnetic particles.

31. A method for isolating a nucleic acid, the method comprising:
(a) contacting a sample comprising the nucleic acid with a probe to form a nucleic acid hybrid, wherein the probe comprises a string of arylboronic acid moieties;
(b) contacting the nucleic acid hybrid of (a) with a solid support having attached thereto a arylboronic acid complexing moiety to form a complex comprising the nucleic acid hybrid and the solid support; and
(c) separating the complex of (b) from the sample.

32. The method of claim 31, further comprising:
(d) washing the complex to remove any uncomplexed sample components.

33. The method of claim 31, wherein the nucleic acid is an RNA or a DNA.

34. The method of claim 31, wherein the nucleic acid is a first strand of a cDNA synthesized using a primer that comprises a string of arylboronic acids.

35. The method of claim 31, wherein the nucleic acid is a product of a polymerase chain reaction or ligase chain reaction in which one or more primers comprises a string of arylboronic acids.

36. The method of claim 31, further comprising:
(d) dissociating the nucleic acid from the complex.

37. A method for purifying a nucleic acid sequencing reaction product, the method comprising:
(a) hybridizing a primer that comprises a string of arylboronic acid moieties to a nucleic acid template to form a template-primer hybrid;
(b) extending the primer by contacting the hybrid with a polymerase in a reaction mixture comprising deoxynucleotides and dideoxynucleotides to form a primer extension product;
(c) contacting the primer extension product of (b) with a solid support having attached thereto a arylboronic acid complexing moiety to form a complex comprising the primer extension product and the solid support; and
(d) separating the complex of (c) from the reaction mixture.

38. The method of claim 37, wherein the nucleic acid sequencing reaction product is obtained by a cycle sequencing reaction (CSR).

39. The method of claim 37, further comprising:
(e) denaturing the complex to release the primer extension products from the nucleic acid template.

40. The method of claim 37, further comprising:
(e) washing the complex to remove any uncomplexed sample components; and
(f) dissociating the primer extension products from the complex.

41. The method of claim 40, wherein the dissociation is effected by elevating the temperature of a liquid that comprises the complex.

42. The method of claim 41, wherein the liquid has an ionic strength of between about zero and about 10 mM.

43. The method of claim 42, wherein the liquid is water.

44. The method of claim 37, wherein the string of arylboronic acid moieties is attached to the 5′ end of the primer.

45. The method of claim 37, wherein a detectable label is attached to the dideoxynucleotides.

46. The method of claim 45, wherein the detectable label is a dye molecule.

47. The method of claim 45, wherein the dideoxynucleotides are one or more of ddA, ddC, ddG and ddT, and wherein a different detectable label is attached to each of the dideoxynucleotides.

48. A method of sequencing a nucleic acid, the method comprising:
analyzing the purified primer extension products of claim 40 by gel electrophoresis or capillary electrophoresis.

49. The method of claim 48, wherein the primer extension products are injected directly onto a capillary electrophoresis column without desalting or concentrating the primer extension products.


SEQUENCE LISTING (5 sequences)

SEQ ID NO: 1 - DNA, 611 bp, Artificial Sequence
  Description of Artificial Sequenceautomated sequencing trace of PBA-4-modified cycle sequencing primer extension products from a 1 kilobase region of pUC18 plasmid DNA
cccgcccccncccctttaccacctttgacctgccagcagcctttggnaacaaggnnagcagagcgaggtatgtaggcggtgctacagagttcttgnagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccntcgtgtanataactaccatacgggagggcttaccatctggccccagtgctgcaatgataccgcgagacccacgctcaccggctccagatt

SEQ ID NO: 2 - DNA, 235 bp, Artificial Sequence
  Description of Artificial Sequenceautomated sequencing trace of PBA-4-modified primer extension products from a 250 base pair PCR product derived from pUC18 plasmid
gcctatatttgccgaatacccaggactacactcaccgagcgacctccctgatacacggtggcaacgccgccttgaatgatcctgatatgacaacaatacgagggccgagtgctctgtgctgcaacttacggattggtccaaaaacacggccgactgtggcagttcctaaataaattattctgccgactccgccgggggggcgggctaactactayaggatctannnnnnnnnnnn

SEQ ID NO: 3 - DNA, 511 bp, Artificial Sequence
  Description of Artificial Sequenceautomated sequencing trace of unmodified primer extension products from a 790 base pair PCR product derived from lambda DNA
naaactcatcaggntcagccagcagcatcagcggtgctgactgaatcatggtgaactcacgcgccggatcgccggtggtcacccagtttttcgggtaacgggcagaggcgttaatgccttcgcgctgtgcgtccgcatcctgaatgcagccataggtgcgcaaaccgcgttgcctgagtgttccccagcaccatcgtgttgtccggcaggaaanttctttttgacgccgttttccacgttactgtccggaaatacacgacaatggccacatcgccatacatccccttataggacaccgctttgcccaggtctttcacgctttctccagctcggaattagagccacaaagggtatccacttctccttaacgctttaaaggaacggaanacccccacctttcggatcaaaacaataatttcnccacacgctngcgttnacgcntagcttcaattctcggtcggtcnacntgaattntcanctgcncatccgtgccccngaatgcnttantttt

SEQ ID NO: 4 - DNA, 525 bp, Artificial Sequence
  Description of Artificial Sequenceautomated sequencing trace of PBA-4-modified primer extension products from a 790 base pair PCR product derived from lambda DNA
tngnaaactcatcggttcagccagcacatcagcgggttctgactgaatcatggtgaactccgcgccggatcgccggtngtcaccagtttttcgggtaacgggcagaggcgttaatgccttcgccgctgtgcgtccgcntcctnaatgcggccataggtgcgcagaccgcggtgcctgagttgtnccccagcaccatcgttgttgtccggcaagaaagttctttttgacgccgttttccacgntactgtccgggaatacacgacnatggccacatcgccatacatccccntataggacaccgctttgcccgggtctttcaccgctgtctccagctcggaaatnanaaccacgaacgggtatccaacttctccttgacggctttgaaaggaacgaacancgcccaccctttcggatcgaacacnataatttcaccacagcgctnggcgttcancgcntaggcttcaatnctcggtcggtcaangtggantnttnncctggctcnntccgtgccccggaatgnntaan

SEQ ID NO: 5 - DNA, 600 bp, Artificial Sequence
  Description of Artificial Sequenceautomated sequencing trace of PBA-4-modified primer extension products from a 790 base pair PCR product derived from lambda DNA
naaatgccttcgcgctgtgcgtccgcatcctgaatgcagccataggtgcgcagaccgcgtgcctgagtgttccccagcaccatcgtgttgtccggcaggaagttctttttgacgccgttttccacgtactgtccggaatacacgacgatggccacatcgccatacatccccttataggacaccgntttgcccaggtctttcaccgctgtctccagctcggaattanagccacgacgggtatccagcttntccttgacggctttgaaggaacggaacagcgcccagcctttcggatcgaacacgatgatattcaccacacccgctggcgttcagcgcgtaggcttcgatatcgttggtcgggtcatacgtggacttgtnacgcttgctccactccgtgccgccggactgngngatgttattttcttnactgggggcccatatccacctnaaccnggatcgaaggcttcaccggcctnggggtattttgccctttaagcacnggaaaaaacggcctgcattttnttttgaccttgagcaaanggccaagcnttttttgncacnccattgttctttgcattgantgantgnccancgggcggg